(19) United States
(12) Patent Application Publication
(10) Pub. No.: US 2002/0081587 A1
(43) Pub. Date: Jun. 27, 2002

(54) Biological microbalance array module chip

(76) Inventors: Jeng-Yang Hwang, Tainan City (TW); Hen-Sung Tsai, Tainan City (TW)

Correspondence Address: BACON & THOMAS, PLLC, 625 Slaters Lane - 4th Floor, Alexandria, VA, 22314-1176, US

(21) Appl. No.: 09/745,934

(22) Filed: Dec. 26, 2000

(51) Int. Cl. C12Q 1/68; C12M 1/34; G01F 1/64

(52) U.S. Cl. 435/6; 435/287.2; 205/777.5

(57) ABSTRACT

A biological microbalance array module chip, comprising a piezoelectric crystal array, probes, and a plurality of electrodes, wherein the probes is an oligonucleotides or immune materials is disclosed. The probes can be immobilized on each electrode on the crystal surface of the piezoelectric crystal array. This biological probe is used to indicate the result of hybridization or immune reaction through detecting the change of oscillation frequency of the crystal probes attached. These piezoelectric crystal can be further arranged in an N×M array. Each piezoelectric crystal is attached with probes that specifically bind to a molecular structure of the target oligonucleotides or immune materials. The biological microbalance array module chip can fast and simultaneously detect hybridization and immune reaction results.

### BACKGROUND OF THE INVENTION

1. Field of the Invention

The present invention relates to a biological microbalance array module chip and, more particularly, to a biological microbalance array chip for DNA, RNA sequence analysis or immunoassay.

2. Description of Related Art

Currently, DNA or RNA sequence analysis techniques are widely applied to many biology related research fields such as disease diagnosis, genetic research and pharmaceutical development. These sequence analysis techniques are important tools for exploring secretes of genes. Generally speaking, two basic procedures such as autoradiography and optical detection were used among these sequence analysis techniques. The oligonucleotide fragments such as DNA fragments or RNA fragments are incorporated with radioactive labels (e.g. P³²) or fluorescent labels before further analysis. The labeled oligonucleotide fragments can be target fragments with unknown sequence or known sequence. After the oligonucleotide fragments are labeled and treated by further analysis procedures (e.g. hybridization of DNA fragments or RNA fragments), results of analysis can be read out through radioactive detection or optical detection. However, several drawbacks are associated with these traditional sequence analysis techniques.

First, prolonged exposure to radioactive elements will increase the risk of acquiring genetic disease (e.g. cancer). Typically, workers are required to be well trained and monitored by devices according to regulation. These protected procedures increase expense cost and time cost. In addition, the incorporation of a radioactive label into nucleic sequence increases the complexity and cost of sequence analysis. Further, additional special hardware (e.g. β counter) required for the radioactive detection also increase the expense significantly.

Similar disadvantages are found in the application of fluorescent dyes in optical detection. Most of fluorescent dyes used for marking oligonucleotide fragments are mutagenic or carcinogenic. Exposure to the fluorescent dyes also increases the risk of acquiring genetic disease. On the other hand, the incorporation of a radioactive label into nucleic sequence increase the complexity and cost of sequence analysis. Additional hardware are also required for the optical detection. Therefore, it is inconvenient to use these DNA or RNA sequence analysis techniques to analyze the molecular structures or sequence of oligonucleotide. A convenient and fast sequence analysis techniques for oligonucleotide is in strong demand now.

On the other hand, it is known that piezoelectric substrates, such as quartz, can mechanically oscillate in a perpendicular or parallel field. The oscillation frequency of the piezoelectric substrates is dependent on the weights of other foreign materials attached on them in a linear relationship. In 1986, Tompson (M. Tompson, C. L. Arthur, and G. K. Dhaliwal, Liquid-Phase piezoelectric and Acoustic Transmission Studies of Interfacial Immunochemistry, Anal. Chem. Vol. 58, pp. 1209, 1986) and Karube (H. Murumatsu, K. Kajiwara, E. Tamiya, and I. Karube, piezoelectric Immuno Sensor for the Detection of Candida Albicans Microbes, Anal. Chem. Acta, Vol. 188, pp. 257-261, 1986) disclosed a quartz crystal microbalance (QCM) acting as a biochemical immuno sensor. Piezoelectric crystal materials are used in hybridization experiments by immobilizing nucleic acids on crystal surface to form a DNA probe (Shuichiro Yamaguchi, Takeshi Shimomura; Adsorption, Immobilization, and Hybridization of DNA Studied by the Use of Quartz Crystal Oscillators. 1993 65:1925-1927. Hongbo Su, Krishna M. R. Kallury and Michael Thompson. Interfacial Nucleic Acid Hybridization Studied by Random Primer P³² Labeling and Liquid-Phase Acoustic Network Analysis. 1994 66:769-777). In 1991, S. P. A. Fodor, etc. developed a technology to parallelly synthesize great number of different oligonucleotide or peptide fragments on a single flat surface (Fodor, S. P. A., J. L. Read, M. C. Pirrung, L. Stryer, A. T. Lu and D. Solas; Light-Directed, spatially addressable parallel chemical synthesis; Science, 1991 251:767-773). It is the major technique for producing the DNA chip. Such DNA chips immobilize DNA fragments with known sequence on the substrate. These immobilized DNA fragments are expected to bind with target DNA fragments marked with fluorescent groups or radioactive isotopes after hybridization. After washing to remove interference of uncombined fragments, the sequence of target DNA fragments detected or identified by autoradiography or optical detection. However, although a large number of oligonucleotide fragments with different or identical sequence can be immobilized or synthesized on a chip simultaneously, the inconvenience and high cost of readout process still remains. This inconvenience also limits the wide application of biological microbalance array chips.

Therefore, it is desirable to provide an improved biological microbalance array chip to obviate the aforementioned problems.

### SUMMARY OF THE INVENTION

The object of the present invention is to provide a biological chip or a biological microbalance array module chip that can fast and simultaneously detect the hybridization results of multiple oligonucleotide fragments without incorporated pretreatment process of oligonucleotide fragments and radioactive elements or fluorescent groups.

Another object of the present invention is to provide a biological chip or a biological microbalance array module chip that can fast and simultaneously detect immune reaction of multiple immune materials without incorporation process of immune materials and marking labels such as radioactive elements or fluorescent groups.

The biological chip of the present invention, comprises a test site formed on a piezoelectric substrate; a plurality of electrodes associated with said test site, said test site having at least two electrodes attached thereto, and at least one surface of said electrodes on said test site being optionally immobilized with a plurality of probes which specifically bind to a target molecular structure; and circuitry which couples to said electrodes of said test site to output frequency variance generated from said test site.

The biological microbalance array module chip of the present invention, comprising a plurality of piezoelectric substrates in an N×M array, wherein each substrate has a test site formed thereon, said N and M are integrals greater than 1; a plurality of electrodes associated with said test sites, each test site having at least two electrodes attached thereto, and at least one surface of said electrodes on said test site being optionally immobilized with a plurality of probes which specifically bind to a target molecular structure; and circuitry which couples to said electrodes of each test site to output frequency variance generated from said test sites; wherein different test sites have probes which specifically bind to different target molecular structures.

The amount of probes which have bound to target molecular structures can be determined by measuring the frequency variation of each test site.

Other objects, advantages, and novel features of the invention will become more apparent from the following detailed description when taken in conjunction with the accompanying drawings.

### BRIEF DESCRIPTION OF THE DRAWINGS

FIG. 1 illustrates the schematic representation of the biological chip or the basic functional unit (or test site) of the biological microbalance array module chip of the present invention.

FIG. 2 illustrates the schematic representation of three general types of the detection methods of the biological microbalance array module chip of the present invention.

FIG. 3 illustrates the biological microbalance array module chip of the present invention.

FIG. 4 illustrates it can use S. P. A. Fodor's manufacturing process on the biological microbalance array module chip of the present invention.

FIG. 5 illustrates it can use S. P. A. Fodor's manufacturing process on the biological microbalance array module chip of the present invention.

### DETAILED DESCRIPTION OF THE PREFERRED EMBODIMENT

The biological chip of the present invention, comprising a test site formed on a piezoelectric substrate; a plurality of electrodes associated with said test site, said test site having at least two electrodes attached thereto, and at least one surface of said electrodes on said test site being optionally immobilized with a plurality of probes which specifically bind to a target molecular structure; and circuitry which couples to said electrodes of said test site to output frequency variance generated from said test site.

The electrodes on each site can be deposited on any portion of the site of the biological chip of the present invention. However, a couple of the electrodes sandwich the test site of the biological chip of the present invention is preferred.

The biological microbalance array module chip of the present invention, comprising a plurality of piezoelectric substrates in an N×M array, wherein each substrate has a test site formed thereon, said N and M are integrals greater than 1; a plurality of electrodes associated with said test sites, each test site having at least two electrodes attached thereto, and at least one surface of said electrodes on each test site being optionally immobilized with a plurality of probes which specifically bind to a target molecular structure; and circuitry which couples to said electrodes of each test site to output frequency variance generated from said test sites; wherein different test sites have probes which specifically bind to different target molecular structures.

The electrodes on each site can be deposited on any portion of each site. A couple of the electrodes which sandwich each test site and at least one surface of the electrodes on each said test site are optionally immobilized by a plurality of probes is preferred. The probes attached on the electrodes are probes that can bind corresponding specific target molecular structures. The probes which have bonded to the molecular structure of target molecules are determined by measuring the variation of the frequency of each test site. The amount of probes which have bonded to the molecular structure of target molecules are determined by measuring the variation of the frequency of each test site.

The piezoelectric substrates of the biological chip or biological microbalance array module chip of the present invention can be any materials with piezoelectric properties. Piezoelectric materials such as quartz, LiNbO₃, NiTaO₃, BaTiO₃, and PbTiO₃ are preferred candidate for the piezoelectric substrates used for the present invention. Most preferably, the piezoelectric substrate is quartz.

The presence and the mass of the target molecular structures that bound to the probes of the biological microbalance array module chip of the present invention can be determined by detecting the changes of frequency of each test site. The principle of the determination of the biological microbalance array module chip is illustrated following.

It is well known that as the weight or mass on a piezoelectric substrate is changed, the frequency of the oscillation of the piezoelectric substrate also changed. The variance of oscillation frequency can be predicted through calculation of known formula. For example, as a piezoelectric substrate, e.g. quartz, is attached by foreign materials, the oscillation frequency of the quartz can be predicted by following formula (I):

ΔF=−2.26×10⁶F²ΔM_{S}/A (I)

wherein ΔF is the variance (Hz) of frequency caused from the change of mass ΔM_{S}; F is the resonant oscillation frequency (MHz) of the piezoelectric substrate; and A is the area (cm²) of the piezoelectric substrate that foreign materials attach on.

For the mass change ranges from ppm to ppb, the formula (I) illustrated above is suitable to predict the change of frequency precisely. The mass of foreign materials bound to the piezoelectric substrates can therefore be calculated according to the variance of frequency. Therefore, the mass of foreign materials bound on the piezoelectric substrate can be qualitatively and quantitatively analyzed by detecting the change of frequency generated from the change of mass of piezoelectric substrates. In other words, as foreign materials attach on the piezoelectric substrates or bind with the probes bound on the piezoelectric substrates, the mass and the result can be detected by measuring the change of frequency of the piezoelectric substrates. If certain probes bound on the piezoelectric substrate are attached by the corresponding target molecules, the oscillation frequency will change as the corresponding target molecules specifically bind or hybridize with the probes on piezoelectric substrates.

The probes bound on the surface of the electrode of each test site can be any probes that can bind (or hybridize) with molecular structures of target molecules specifically. For example, the probes can be antibodies, antigens, polypeptides, receptor proteins, DNA fragments, RNA fragments or synthesized oligonucleotides. Preferably, the probes are probes binding to a DNA molecule, probes binding to an RNA molecule, probes bind to a cell, or probes binding to an antibody. On the other hand, electrodes on each test site can cover either full area or one part of area of each test site. The patterns of the electrodes on each test site are not limited. The electrodes deposited on each test site can be made by any conductive materials. Preferably, the electrodes are metals. Most preferably, the electrode is gold or silver.

The circuitry of the biological microbalance array module chip coupled to the electrodes of each test site on the piezoelectric substrate is to output frequency signals. However, circuitry containing at least one amplifiers, at least one counters and optionally a multiplexer is preferred to couple to the biological microbalance array module chip of the present invention to detect which test site transmits frequency signals out or how much the frequency changes. The amplifiers on the circuitry can be either amplifiers foreign to each test site or amplifiers built in each test site (i.e. built-in amplifiers). Optionally, the amplifiers built in the test site can be arranged on the same integrated circuit chip where the biological microbalance array module chip locates on through known VLSI or ULSI technology. The built-in amplifiers on each test site can be arranged in any part of the test site to amplify the frequency signal in situ. One of the obvious advantage of the built-in amplifiers of each test site is to reduce the interference of noise in situ effectively. Preferably, the built-in amplifiers are arranged below each test site through VLSI or ULSI technology. The counters can be either foreign counters or amplifiers built in each test site (i.e. built-in counters). The counters either foreign to the test site or built in the same integrated circuit chip of the test site can be used for the biological microbalance array module chip. For the counters built in the same integrated circuit chip of the test site, the counters can be arranged in any part of each test site to detect the frequency signal in situ. Preferably, the built-in counters are arranged below each test site through VLSI or ULSI technology. For the counters built in each test site, the interference of noise will be reduced effectively.

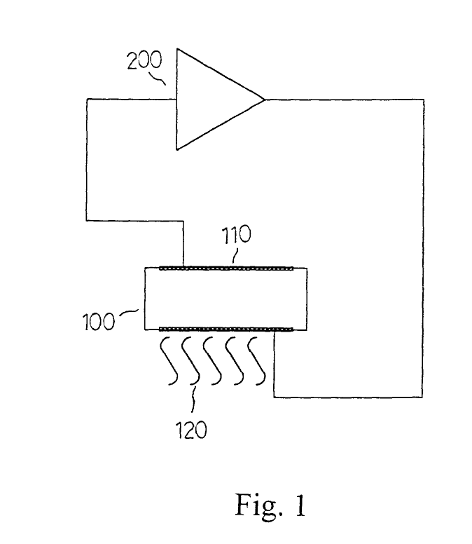

The preferred embodiment of the present invention and its advantages are best understood by referring to FIGS. 1 to 5 of the drawings, like numerals being used for like and corresponding parts of the various drawing.

FIG. 1 illustrates a basic functional unit of the preferred embodiment of the present invention used for DNA or RNA sequence determination or immunoassay.

The basic functional unit, biological chip 100, of the present invention comprises a piezoelectric substrate 140, probes 120 with known sequences or known immune materials (e.g. antibodies) attached on the electrodes 10 of the test site on the piezoelectric substrate 140. The electrodes 110 are deposited on the test site of the piezoelectric substrate 140. The piezoelectric substrate 140 acts as a substrate for the attachment of probes and oscillation source.

In operation, samples containing specific target molecular structures that can bind with the probes on the biological chip of the present invention are passed or placed over the biological chip of the present invention 100. For example, samples containing specific oligonucleotide fragments such as DNA fragments or RNA fragments with unknown sequences are passed or placed over the biological functional unit, the biological chip 100 of the present invention. Once the molecules with target molecular structures in the sample hybridize with the probes 120 on the electrode 110, the hybridization (or combination) between molecules with target molecular structures and the probes increases the weight on the electrode surface of the functional unit 100. This increase of the mass further causes a variation of the oscillation frequency of functional unit 100. In most cases, as the specific DNA fragments or the specific RNA fragments in the sample hybridize with the probes, the combination between target fragments and the probes will cause a variation of the oscillation frequency of functional unit 100. The functional unit 100 later transmits frequency-dependent electric signals to foreign circuit. The signals are amplified through amplifier 200. The amplified signals can be further detected and read out. Therefore, the amounts and the results of hybridization can be determined through the variation of frequency signals.

On the other hand, a plurality of functional units of the present invention depicted above can be arranged in an N×M array and connect by a circuit to form a biological microbalance array module chip. Each individual functional unit (i.e. test site) can be immobilized with specific probes (e.g. probes with specific known sequence of oligonuleotides or immune materials) for applications. When samples which contain specific molecular structures of target molecules (e.g. oligonucleotide fragments or immune materials) are laid on or passing through the module chip of the present invention, the module chip can determine which unit (or test site) in the array is bound (or hybridized) and how many probes on the unit (or test site) are bound (or hybridized). In other words, the module chip can detect the presence and the amount of various target molecules with target molecular structures (e.g. oligonucleotide fragments or immune materials) at the same time.

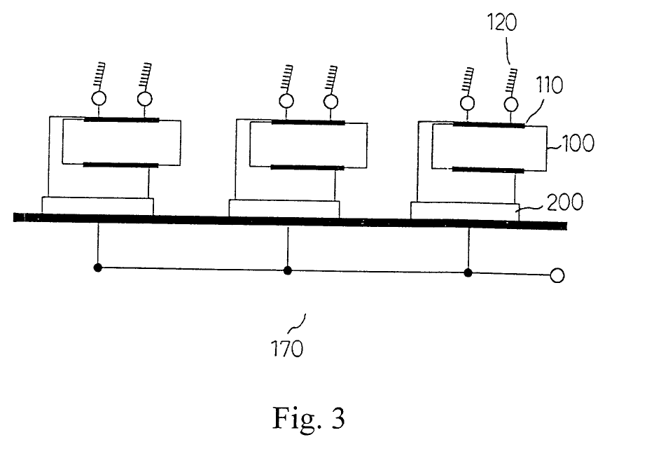

FIG. 3 illustrates another preferred embodiment of the present invention. The biological microbalance array module chip of the present invention comprises an array of test sites and detection circuitry. The array of test sites contains N×M (e.g. 4×2) test sites wherein N and M are integrals equal or greater than 1. The array of test sites is formed on a piezoelectric crystal substrate array. Each test site contains probes immobilized on the surface of said test site on the piezoelectric crystal and a couple of electrodes associated with the test sites. The probes 120 (e.g. oligonucleotides with known sequences or immune materials) attached on the surface of the test sites on the piezoelectric substrate 140 (e.g. quartz) can be immobilized through any known methods. In the embodiment in FIG. 3, the probes 120 are immobilized by spatially addressable immobilization. The electrodes 10 are deposited on the test sites on the piezoelectric substrate 140. The circuitry connects to the coupled electrodes of the test sites on the piezoelectric substrate substrate. In this embodiment, the circuitry connects to built-in amplifiers, built-in counters and a multiplexer to detect which test site on the substrate transmits electric signals out and how much the frequency changes. The built-in counters are arranged on each test site to detect the change of oscillation frequency. Therefore through the corporation of amplifiers, counters and the multiplexer, the frequency signals can be recognized and read out to determine the probes on which test site have bound or hybridized to an associated molecular structure of target molecules.

The test sites of the biological microbalance array module chip can be formed and separated to each other by photolithography or cutting. Electrodes of each test sites can be deposited on the test sites of biological microbalance array module chip through any known prior arts such as coating, vapor deposition, lithography or sputtering. Preferably, the electrodes are made of metals. Most preferably, the material of the electrodes is gold or silver. The probes on the test sites can be immobilized or synthesized through spatially addressable immobilization or other known conventional methods. Any prior art for immobilizing the probes can be applied here. For example, various probes such as oligonucleotides can be immobilized through offset printing by a robotic x-y table on a piezoelectric crystal to form a biological microbalance array module chip. Preferably, the probes can be immobilized by spatially addressable immobilization.

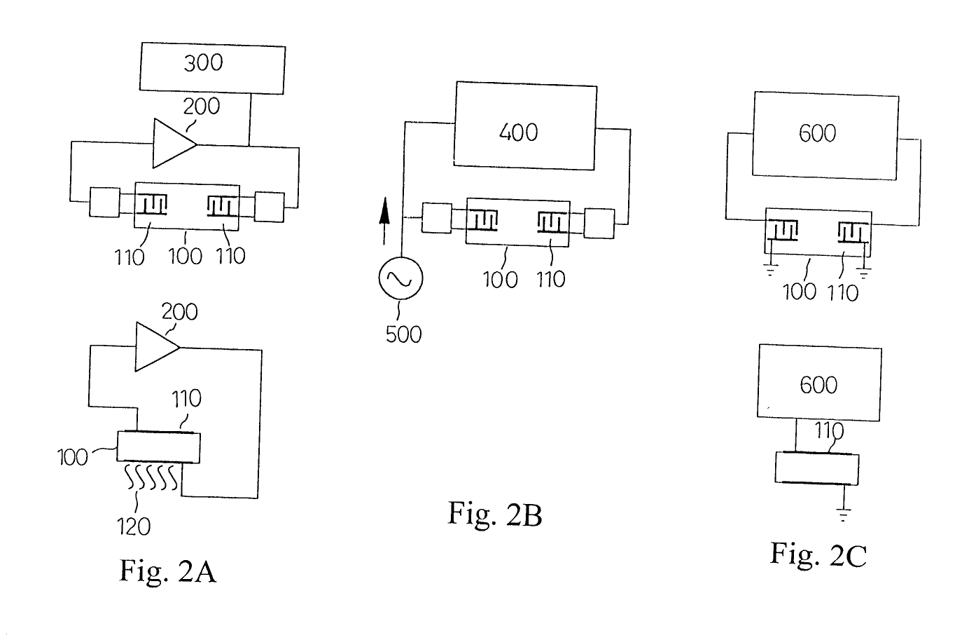

FIG. 2A to 2C illustrate a preferred embodiment of methods or circuit to detect the variation of the frequency signals produced from the biological microbalance array module chip of the present invention.

The detection method using the circuit illustrated in FIG. 2A is a passive detection method for detecting the presence of biological polymers. The biological microbalance array module chips are connected to a foreign circuit containing amplifiers. A frequency counter connects to the circuit to detect amplified frequency signals. As target oligonucleotide fragments hybridize with the probes on the biological microbalance array module chip of the present invention, a variation signal of frequency caused by changes of masses on the biological microbalance array module chip will output and transmit to the circuit and amplified by the amplifier on the circuit. This frequency signal is then detected and recognized by counter 300 of the circuit.

FIG. 2B illustrates another circuit used for detecting the frequency variation from the biological microbalance array module chip. While the target oligonucleotide fragments hybridize with the probes on the biological microbalance array module chip of the present invention, the target oligonucleotide fragments will combine with the probes on the biological microbalance array module chip and therefor change the masses of the materials attached on the biological microbalance array module chip. As an external AC source is applied on the circuit, the frequency change of the biological microbalance array module chip can be detected by a counter 400. This is the active detection method. The detection method can be any kind of types that can detect the variation of frequency of the biological microbalance array module chip (e.g. FIG. 3).

The circuit used here to detect or read out the result of hybridization can be a foreign circuit outside the biological microbalance array module chip. On the other hand, the circuit to detect or read out the result of hybridization can also be integrated on the piezoelectric crystal that the test site of the biological microbalance array module chip locates on. In other words, the circuit of the amplifier and the frequency counter for detecting and recognizing the variation of frequency can be formed on the same piezoelectric crystal that the test site locate on and the circuit can be designed to connect the test sites of the biological microbalance array module chip.

The spatially addressable immobilization method here used is a method provided for immobilizing oligonucleotides on predefined regions of a surface of a solid support. Although the spatially addressable immobilization method is not the major key point of the present invention, the illustration following is included here for clear understanding. The methods include coating a layer of compounds with photochemically protected functional groups (e.g. protected thiols) to the surface of the solid support. The photochemically protected functional groups (e.g. photochemically protected thiols) which are inert to oligonucleotides can be converted into functional groups (e.g. thiols) reactive to oligonucleotides by irradiation. Therefore, the oligonucleotide can be immobilized to the surface of predefined area by selective activation through irradiation on the selective surface of the solid support.

The spatially addressable immobilization method also makes forming patterns with the same reactivity to nucleotides possible. For example, by using conventional lithography, light can be projected to relatively small areas on the surface precisely. Thus, the spatially addressable immobilization method can be used to activate discrete, predetermined locations on the surface for attachment of various oligonucleotide. The resulting surface can be used for many various applications. For example, samples containing different oligonucleotides can be analyzed qualitatively and quantitatively through direct binding assays at the same time. The affinity and the number of the oligonucleotides in the sample can be detected or analyzed by the oligonucleotide probes attached to the surface of the solid support.

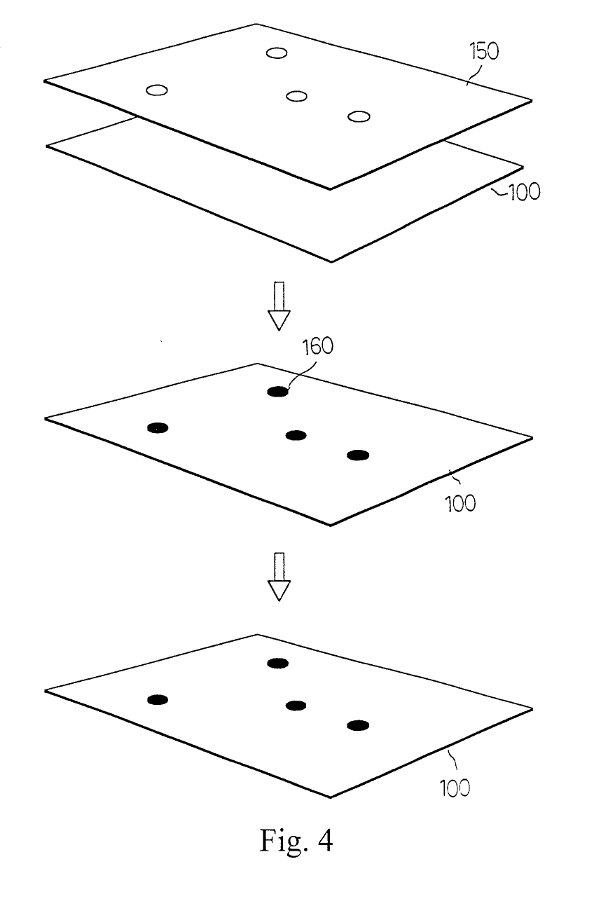

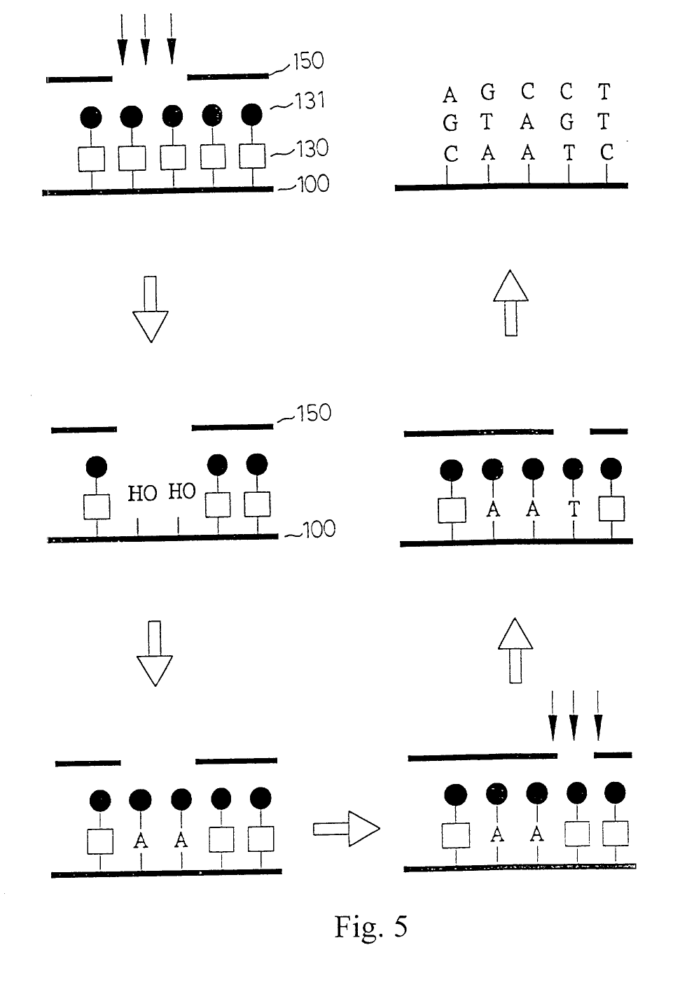

S. P. A Fodor's spatially addressable immobilization process can be used in the present invention is illustrated in FIG. 4 and 5. The locations to be bound with specific nucleic acids are exposed to a radiation with specific wavelength. The selective locations or areas to be exposed are controlled by a series of masks. The protected groups bound with the functional groups attached on the selective exposed areas or locations leave after irradiation and the deprotective functional groups attached on the selective exposed areas or locations become reactive. Then the reactive functional groups attached on the selectively exposed areas or locations further react with specific nucleic acids added subsequently to form immobilized nucleic acids. On the other hand, no deprotection occurs in the unexposed areas or locations. This means that no reaction occurs in the unexposed areas or locations. Immobilized oligonucleotides with specific sequences can be formed through the processes illustrated above. The processes are repeated with designed masks and designed nucleic acids. Specific oligonucleotides with designed subsequences can be immobilized and synthesized on the substrate through designed masks and designed nucleic acids. A substrate with immobilized oligonucleotides having various or identical specific sequences can be formed after several designed exposure and reaction processes.

The target oligonucleotide fragments can be any DNA fragments or RNA fragments (natural or artificial). The target DNA fragments or RNA fragments can even come from living species or dead cells. The method to detect the biological microbalance array module chip of the present invention can be applied in the field of medical diagnosis, genetic research or pharmaceutical applications. Through the assistance of the detection of the biological microbalance array module chip of the present invention, the results of the hybridization of probes and target oligonucleotides can be recognized very fast. The probes can be designed to hybridized with the DNAs or RNAs of pathogenic microorganisms. Then the biological microbalance array module chip of the present invention can be easily used for the medical diagnosis through the detection of the presence of the DNAs or RNAs of pathogenic microorganisms. Even the variety and number of pathogenic microorganisms can be determined simultaneously through the readout of the result of hybridization. Since the biological microbalance array module chip of the present invention can detect and read out the multiple results of hybridization of probes and target biological polymers, the biological microbalance array module chip of the present invention can also be applied to the development of new medicines. For example, the probes such as genes of pathogenic microorganisms or molecules for transmitting messages between cells are designed to be immobilized on biological microbalance array module chip, then the candidate molecules for pharmaceutical purpose are passed or laid on the biological microbalance array module chip. The results of the hybridization between probes and candidate molecules can be read out fast and are helpful to screen out good or proper candidate molecules for pharmaceutical purposes.

Although the present invention has been explained in relation to its preferred embodiment, it is to be understood that many other possible modifications and variations can be made without departing from the spirit and scope of the invention as hereinafter claimed.

What is claimed is:

1. A biological chip, comprising:
a test site formed on a piezoelectric substrate;
a plurality of electrodes associated with said test site, said test site having at least two electrodes attached thereto, and at least one surface of said electrodes on said test site being optionally immobilized with a plurality of probes which specifically bind to a target molecular structure; and
circuitry which couples to said electrodes of said test site to output frequency variance generated from said test site.

2. A biological microbalance array module chip, comprising:
a plurality of piezoelectric substrates in an N×M array, wherein each substrate has a test site formed thereon, said N and M are integrals greater than 1;
a plurality of electrodes associated with said test sites, each test site having at least two electrodes attached thereto, and at least one surface of said electrodes on each test site being optionally immobilized with a plurality of probes which specifically bind to a target molecular structure; and
circuitry which couples to said electrodes of each test site to output frequency variance generated from said test sites;
wherein different test sites have probes which specifically bind to different target molecular structures.

3. The biological microbalance array module chip as claimed in claim 2, further comprising circuitry for detecting said frequency variance generated from said test sites to determine which probes have bound to an associated target molecular structure by measuring said frequency variance generated from said test sites.

4. The biological microbalance array module chip as claimed in claim 3, further comprising circuitry for detecting said frequency variance generated from said test sites to determine how many probes have bound to an associated target molecular structure by measuring said frequency variance generated from said test sites.

5. The biological microbalance array module chip as claimed in claim 2, wherein each test site further comprising an amplifier coupling to said circuitry for amplifing said frequency variance generated from said test sites.

6. The biological microbalance array module chip as claimed in claim 5, wherein each test site further comprising a counter coupling to said circuitry for measuring said frequency variance generated from said test sites.

7. The biological microbalance array module chip as claimed in claim 2, wherein said piezoelectric substrate is quartz.

8. The biological microbalance array module chip as claimed in claim 2, further comprising at least a multiplexer coupling to said circuitry for selecting frequency variance generated by one of said plurality of test sites to output.

9. The biological microbalance array module chip as claimed in claim 2, wherein said circuitry couples to a foreign amplifier.

10. The biological microbalance array module chip as claimed in claim 9, wherein said circuitry couples to a foreign counter.

11. The biological microbalance array module chip as claimed in claim 2, wherein one or more of said probes bind to a DNA molecule.

12. The biological microbalance array module chip as claimed in claim 2, wherein one or more of said probes bind to an RNA molecule.

13. The biological microbalance array module chip as claimed in claim 11, wherein one or more of said probes comprises oligonucleotide.

14. The biological microbalance array module chip as claimed in claim 12, wherein one or more of said probes comprises oligonucleotide.

15. The biological microbalance array module chip as claimed in claim 2, wherein one or more of said probes bind to an antigen.

16. The biological microbalance array module chip as claimed in claim 2, wherein one or more of said probes bind to an antibody.

17. The biological microbalance array module chip as claimed in claim 16, wherein said probes comprise peptide probes.

18. The biological microbalance array module chip as claimed in claim 17, wherein said probes comprise peptide probes

19. The biological microbalance array module chip as claimed in claim 2, which is used for quantitative analysis of target molecules.

20. The biological microbalance array module chip as claimed in claim 2, which is used for indicating species of pathogenic microorganism in medical diagnosis.

21. A method for identifying or measuring molecular structures within a sample substance, comprising following steps:
(a) providing a microbalance array module chip as claimed in claim 2;
(b) providing sample substance on said microbalance array module chip; and
(c) detecting the frequency variation generated from each test site of said microbalance array module chip.

22. A method as claimed in claim 20, wherein at least one of said probes bind to a DNA molecule.

23. A method as claimed in claim 20, wherein at least one of said probes bind to an RNA molecule.

24. A method as claimed in claim 20, wherein at least one of said probes bind to a cell.

25. A method as claimed in claim 20, wherein at least one of said probes bind to an antibody.

26. A method as claimed in claim 20, wherein at least one of said probes bind to an antigen.

27. A method as claimed in claim 20, wherein at least one of said probes comprise peptide probes.
